(19) United States
(12) Patent Application Publication
(10) Pub. No.: US 2009/0215039 A1
(43) Pub. Date: Aug. 27, 2009

(54) INVESTIGATIONS

(75) Inventors: James Curran, Birmingham (GB); Christopher Triggs, Birmingham (GB)

Correspondence Address: MERCHANT & GOULD PC, P.O. BOX 2903, MINNEAPOLIS, MN, 55402-0903, US

(73) Assignee: Forensic Science Service Ltd., Solihull (GB)

(21) Appl. No.: 11/909,052

(22) PCT Filed: Mar. 20, 2006
(86) PCT No.: PCT/GB06/00992

§ 371 Date: Feb. 21, 2008

(30) Foreign Application Priority Data
Mar. 18, 2005 (GB) 0505748.4

(51) Int. Cl. C12Q 1/68

(52) U.S. Cl. 435/6

(57) ABSTRACT

A method of investigation a sample is provided, the sample being a mixture of DNA arising from more than one source. The method includes analysing the sample to obtain a genotype for the DNA present in the sample and assigning a prior probability distribution to the genotype. The likelihood function is considered and a posterior probability distribution for the genotype is established. In this way a probabilistic assessment of the genotype of the major or minor contributor to the sample can be obtained. This is beneficial over prior methods which use a deterministic method, and so involve the use of rule based methods.

This invention is concerned with improvements in and relating to investigations, in particular, but not exclusively in relation to investigations of the genotype and/or mixture proportion of a sample of DNA.

When a DNA profile is obtained from a mixture, it is desirable to be able to establish the likely genotype behind the profile and/or the mixture proportion thereof. With existing approaches, the investigation is deterministic in its nature. As such a series of fixed, definite rules are applied.

The present invention has amongst its aims to provide improved investigations into genotypes and mixture proportion investigations.

According to a first aspect of the invention we provided a method of investigating a sample, the sample being a mixture of DNA arising from more than one source, the method including:

analysing the sample to obtain indications of the DNA present in the sample;

assigning a prior probability distribution to the indications;

considering the likelihood function;

establishing a posterior probability distribution for the indications.

According to a second aspect of the invention we provide a method of investigating a sample, the sample being a mixture of DNA arising from more than one source, the method including:

analysing the sample to obtain a genotype for the DNA present in the sample;

assigning a prior probability distribution to the genotype;

considering the likelihood function;

establishing a posterior probability distribution for the genotype.

According to a third aspect of the invention we provided a method of investigating a sample, the sample being a mixture of DNA arising from more than one source, the method including:

assigning a prior probability distribution to the genotype obtained from analysis;

considering the likelihood function;

establishing a posterior probability distribution for the indications.

According to a fourth aspect of the invention we provided a method of investigating a sample, the sample being a mixture of DNA arising from more than one source, the method including:

analysing the sample to obtain indications of the DNA present in the sample;

establishing one or more possible genotypes for the DNA sample;

establishing a probabilistic measure of the possible genotype being the genotype of the sample; and

considering only those of the one or more possible genotypes for the DNA sample which have a probabilistic measure beyond a threshold against one or more records of genotypes, such as a database.

According to a fifth aspect of the invention we provided a method of investigating a sample, the sample being a mixture of DNA arising from more than one source, the method including:

analysing the sample;

assigning a prior probability distribution;

considering the likelihood function;

establishing a posterior probability distribution for the indications.

The first and/or second and/or third and/or fourth and/or fifth aspects of the invention may include features, options and possibilities from amongst the following.

Preferably the considering of the likelihood function includes its evaluation. The considering of the likelihood function and/or the establishment of the factors involved in the likelihood function may use a graphical model.

The likelihood function and/or graphic model of the likelihood function may include a goodness of fit function or statistic, and in particular a χ² distribution. The likelihood function and/or graphical model of the likelihood function may include information on the mixing proportion and/or genotype combination for the major and minor contributors.

Preferably the prior probability distribution and the likelihood function is used to establish the posterior probability distribution

Preferably the posterior probability distribution informs on the probability of one or more indications or genotypes. The posterior probability distribution may inform by sampling the posterior probability distribution. The distribution of the sample values obtained by sampling may be used to inform on the indication and/or genotype. The sampling may be provided by a Monte Carlo Markov Chain method. The sampling may be provided by a Metropolis-Hastings MCMC sampler.

The sampling may be performed on the full posterior probability distribution of the indications/genotypes and/or mixing proportions and/or associated hyper-parameters.

The method may provide probabilistic assessments on the genotype of the major or minor contributor to be made. The method may provide posterior probability assessments of the most probable genotypes and/or a likely range for the mixing proportion.

The graphic model of the likelihood function may be for a two person mixture. The graphic model may be for a three or more person mixture.

The graphical model may have two components, nodes representing variables and/or directed edges which represent the direct influence of one node on another. The nodes may include one or more of starting nodes, parent nodes, child nodes, constant nodes, stochastic nodes. Nodes may be either constant or be stochastic nodes. The direct edges preferably extend between nodes. Preferably the model can include direct edges which extend from a parent node to a child node. Preferably the model cannot include direct edges returning to a starting node.

Preferably constant nodes are fixed in the graphical model and/or are always founder nodes. Constant nodes may have child nodes, but preferably do not have parent nodes. Stochastic nodes are preferably variables and/or may be given a distribution. Stochastic nodes may have child nodes and/or parent nodes.

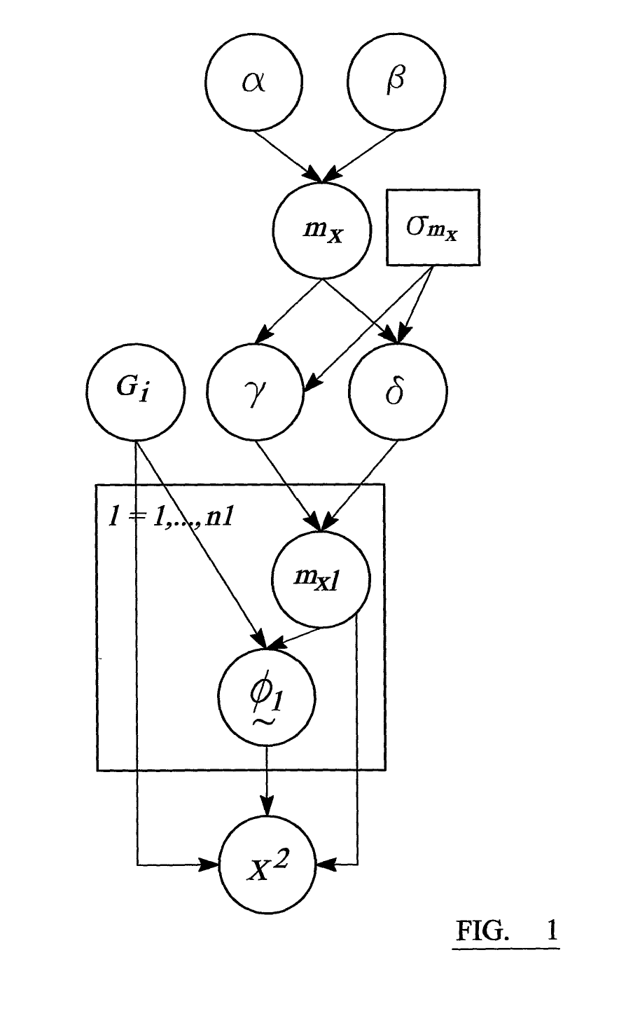

Preferably the invention provides a graphic model substantially as illustrated in FIG. 1. The graphic model may be as illustrated in FIG. 1 with the constant nodes potentially shown as rectangles and/or the stochastic nodes potentially shown as circles.

The graphical model may include one or more of the following possibilities or options set out within the number possibilities:
1. the graphical model includes parameters α, β, which are preferably hyper-parameters of a beta distribution placed on mₓ. where mₓ is the global mixing proportion, and/or may have a or each may have a prior probability distribution which is a Gamma prior, potentially with shape parameter 1 and/or scale parameter 1,000, i.e. α, β˜Γ(1,1000).
2. the graphical model includes parameter mₓ, the global mixing proportion, where, potentially, 100(1−mₓ)% of the mixture comes from the major contributor and 100mₓ% comes from the minor contributor and/or may have a prior probability distribution which is a beta distribution and/or is possibly used to model mₓ, potentially with parameters α and β, i.e. mₓ˜βeta(α,β) and/or with mₓ is scaled between 0.02 and 0.48.
3. the graphical model includes parameter σₘ_{ₓ}, the standard deviation of the locus mixing proportion, with potentially the standard deviation on a given mixing proportion being about 3.5%, so potentially with σₘ_{ₓ} fixed at 0.035.
4. the graphical model includes parameter γ, δ, which are preferably the parameters of a beta distribution for the locus specific mixing proportion mₓₗ, potentially with the quantities determined by the values of mₓ and αₘ_{ₓ} in the following way, for a given value of mₓ, qᵤₚₚₑᵣ=mₓ+2.32σₘ_{ₓ} is calculated, a golden section search may then be used to find γ and δ and/or such that the mode of the beta distribution with these parameters is mₓ and/or the 0.99 quantile of the distribution is qᵤₚₚₑᵣ
5. the graphical model includes parameter mₓₗ, the locus specific mixing proportion, where potentially l=1, . . . , nₗ and nₗ is the number of loci and/or the mixing proportion is allowed to vary from locus to locus according to a beta distribution, potentially with parameters γ, δ, i.e. mₓₗ˜βeta(γ,δ).
6. the graphical model includes parameter Gᵢ, the genotype of the major and the minor contributor, potentially with Gᵢ as a 4×n_{L} array with each row consisting of four integers ranging from 1 to 4, the range of the integers depending on the number of peaks observed at the locus, and/or the distributions for Gᵢ are locus specific and/or dependent on the number of peaks observed at that locus and/or uniform probability is assigned to each combination of peaks and/or this means the prior distribution for Gᵢ is a discrete uniform prior over the space of allowable combinations.
7. the graphical model includes parameter {tilde under (φ)}ₗ, the observed peak area(s) (or height(s)) at a locus, potentially where {tilde under (φ)}ₗ is a vector of length and/or it is assumed that {tilde under (φ)}ₗ has a multivariate Normal distribution (MVN) perhaps with mean vector

$${{μ}_{l}}_{\sim} = \frac{{φ}_{l •}}{2} {( {m}_{xl} , {m}_{xl} , 1 - {m}_{xl} , 1 - {m}_{xl} )}^{′}$$

and/or perhaps with diagonal covariance matrix Σₗ=σₗ²I₄ potentially where

$${φ}_{l •} = {\sum }_{i} {φ}_{li}$$

and/or the value of σₗ² is not important as this because it is not used direction and/or the assumption allows the assumption of a χ² distribution for X²
8. the graphical model includes parameter X², the chi-squared distance of the observed data from expectation under the assumption that the mixing proportion for each locus is known and/or

$${X}^{2} = {\sum }_{l = 1}^{{n}_{l}} {\sum }_{i = 1}^{4} \frac{{( {φ}_{{lG}_{li}} - {E}_{li} )}^{2}}{{E}_{li}}$$

potentially where φ_{lG}_{ᵢ} is the peak at the lth locus to which the major contributor is assumed to have contributed to for i=1, 2 and/or to which the major contributor is assumed to have contributed to for i=3, 4 and/or Eₗᵢ is the expected peak area which is calculated as

$${E}_{li} = E [ {φ}_{li} \vert {m}_{xl} , {G}_{li ; minor} ] = \frac{{m}_{xl} {φ}_{l •}}{2}$$

for the each of the minor peaks and/or

$${E}_{li} = E [ {φ}_{li} \vert {m}_{xl} , {G}_{li ; major} ] = \frac{( 1 - {m}_{xl} ) {φ}_{l •}}{2}$$

for the major peaks

The likelihood function and/or graphical model may be provided on the basis that the true genotype is independent of any of the factors under consideration in the model; and/or the mixing proportion for all the loci, m, depends only on two hyper-parameters α and β; and/or the mixing proportion at each locus is conditionally dependent on two parameters γ and δ which are dependent on the mixing proportion for all loci, mₓ, the standard deviation of a mixing proportion at a locus; and/or the observed peak areas are dependent on the genotype and the mixing proportions at each locus; and/or the κ² statistic X² is dependent on the peak areas and the mixing proportions at each locus. The likelihood function and/or graphical model may be provided on the basis that there is an overall mixing proportion, with conditionally independent mixing proportions at each locus; and/or the priors placed on each genotype are assumed to be uniform; and/or the peak area is evaluated via a chi-squared distribution.

Preferably the method, preferably the likelihood function and/or graphical model, includes a model of the distribution of the distance measure, preferably Euclidean distance, between the expected and observed information, such as peak areas and/or peak heights, for the indications and/or genotypes.

The method, particularly the likelihood function and/or graphical model, may include modelling the peak areas and/or heights at a position, such as a locus, by a multivariate Normal (MVN) distribution. Preferably the distribution has a mean vector:

$${μ}_{\sim} = \frac{{φ}_{•}}{2} {( {m}_{x} , {m}_{x} , 1 - {m}_{x} , 1 - {m}_{x} )}^{′}$$

and/or diagonal covariance matrix:

Σ=σ²I₄.

Preferably the likelihood function and/or graphical model includes the function and/or statistic:

$${X}^{2} = {\sum }_{i = 1}^{4} \frac{{( {φ}_{i} - {E}_{i} )}^{2}}{{E}_{i}}$$

and/or may have a χ² distribution with 3 degrees of freedom. The likelihood function and/or graphical model may account for multiple loci. The function and/or statistic may have 4nₗ−l degrees of freedom where l is the number of loci.

The likelihood function and/or graphical model may provided the joint density function, for a node set V, as being expressed as

$$p ( {x}_{\sim} ) = {\prod }_{v ∈ V} p ( {x}_{v} \vert {x}_{pa ( v )} )$$

The method may include providing the joint density probability as being expressed as a product of the conditional densities of each variable given their parents in the graph.

The likelihood function and/or graphic model may provide that the joint density is:

$$f ( {X}^{2} , {φ}_{\sim} , {{m}_{\sim}}_{xl} , γ , δ , {m}_{x} , α , β , {G}_{i} ) = f ( {X}^{2} \vert {φ}_{\sim} , {{m}_{\sim}}_{xl} , {G}_{i} ) {\prod }_{l = 1}^{{n}_{l}} f ( {m}_{xl} \vert γ , δ ) f ( {m}_{x} \vert α , β ) f ( {G}_{i} ) f ( α ) f ($$

The method may include consideration of the fall posterior probability distribution, but more preferably the conditional density of the genotypes is considered.

The method may include the use of Bayes Theorem and/or may include the calculation:

$$f ( {G}_{i} , {m}_{x} , {θ}_{\sim} \vert {φ}_{\sim} ) = \frac{f ( {G}_{i} , {m}_{x} , {θ}_{\sim} , {φ}_{\sim} )}{f ( {φ}_{\sim} )}$$

where {tilde under (θ)}=({tilde under (m)}ₓₗ, γ, δ, α, β).

The equation may be defined as:

$$f ( {G}_{i} , {m}_{x} , {θ}_{\sim} \vert {φ}_{\sim} ) = \frac{f ( {φ}_{\sim} \vert {G}_{i} , {m}_{x} , {θ}_{\sim} ) f ( {G}_{i} , {m}_{x} , {θ}_{\sim} )}{\int f ( {φ}_{\sim} \vert {G}_{i} , {m}_{x} , {θ}_{\sim} ) f ( {G}_{i} , {m}_{x} , {θ}_{\sim} ) \cdot d {φ}_{\sim}}$$

Preferably the integral on the denominator of the above equation is estimated with one or more Markov-Chain Monte Carlo (MCMC) methods and preferably by a Metropolis-Hastings sampler. Preferably the method provides a method for sampling from the posterior distribution of Gᵢ, mₓ, {tilde under (θ)}, given the data, {tilde under (φ)}. The method may include a sampler, potentially for one locus, which is defined as follows:

1. Randomly select an initial genotype G, and an initial mixing proportion mₓ

2. Calculate the log likelihood, l(G,mₓ|{tilde under (φ)}) using G and mₓ

3. Repeat the following steps a plurality of time
a. Select a new genotype G′, and a new mixing proportion mₓ′
b. Calculate the log likelihood, l′(G′,mₓ′|{tilde under (φ)}) using G′ and mₓ′
c. Generate u˜U[0,1]
d. If log (u)≦min(0,l′(G′,mₓ′|{tilde under (φ)})−l(G,mₓ|{tilde under (φ)})) then
   i. store G′ and mₓ′
   ii. let G=G′, mₓ=mₓ′, and l(G,mₓ|{tilde under (φ)})=l′(G′,mₓ′|{tilde under (φ)})

The sampling may give rise to a stored sample. The method preferably includes the posterior probability density of the mixing proportion being estimated. This may be by means of a density estimate of the stored values of mₓ and/or the posterior probability of the genotypes estimated by counting how many times each one occurred.

The method may be applied to multiple loci. The method may include extra terms in the graphical model. The sampling and particularly the Metropolis-Hastings sampler may provide a method for sampling from the posterior distribution of Gᵢ, mₓ, {tilde under (m)}ₓₗ, γ, δ, α, β, given the data, {tilde under (φ)}. A generalized sampler for multiple loci locus may be defined as follows:
1. Randomly select an initial genotype G, and initial values for mₓ, {tilde under (m)}ₓₗ, γ, δ, α and β
2. Calculate the log likelihood, l(G,{tilde under (θ)}|{tilde under (φ)}) where {tilde under (θ)}=(mₓ, {tilde under (m)}ₓₗ, γ, δ, α, β)
3. Repeat the following steps a plurality of time
   a. Select a random locus l,l˜U [1, . . . , n_{L}]
   b. Select a new genotype Gₗ′ at locus l, to give a new genotype G′
   c. Select new values for {tilde under (θ)}′=(mₓ′, {tilde under (m)}ₓₗ′, γ′, δ′, α′, β′)
   d. Calculate the log likelihood, l′(G′,{tilde under (θ)}′|{tilde under (φ)})
   e. Generate u˜U[0,1]
   f. If log (u)≦min(0,l′(G′,{tilde under (θ)}′|{tilde under (φ)})−l(G,{tilde under (θ)}|{tilde under (φ)})) then
      i. store G′ and {tilde under (θ)}ₓ′
      ii. let G=G′, {tilde under (θ)}={tilde under (θ)}′, and l(G,{tilde under (θ)}|{tilde under (φ)})=l′(G′,{tilde under (θ)}′|{tilde under (φ)})

The sampling may give rise to a stored sample.

The sampling may be performed until 10,000 or more proposals have been accepted, more preferably at least 50,000 proposals and ideally at least 90,000 proposals. The sampling may discard some of the iterations, for instance the first 7,500 iterations. The sampling may take 1 proposal in every n proposals, where n is between 2 and 15, preferably 9. The sampling may continue until a final sample size of at least 1000, more preferably at least 5,000 and ideally at least 10,000 is reached.

From the posterior probabilities, those genotypes above a threshold probability may be selected, for instance selected as likely. The threshold may be combinations which are no more than 10 times less likely than the most likely.

The method may provide a method for probabilistically resolving mixed DNA profiles into a major and minor component. Preferably the method is set up in a Bayesian framework and/or allows inferences about the parameters which are believed to drive the mixing process to be made and/or allows a probabilistic assessment of the genotypes to be produced.

The method may include within the likelihood function and/or graphical model factors for heterozygous balance. The method may be used to simulate a probability density function for heterozygous balance. The method could also be extended to include one or more stutter, preferential amplification, artefacts and more than two contributors to the mixture within the model.

The results of the analysis may be expressed in terms of continuous information and particularly continuous quantitative data. The results of the analysis may include peak area and/or peak height information, particularly in respect of allele size.

The method is preferably not a deterministic method. The method is preferably not a rule based method and/or rule based optimization.

Preferably the method ranks the information from the investigation and/or assesses the worth of the information from the investigation and/or informs on the worth of the information.

Various embodiments of the invention will now be described, by way of example only, and with reference to the accompanying drawings, in which:

FIG. 1 is a graphical model for a two person mixture;

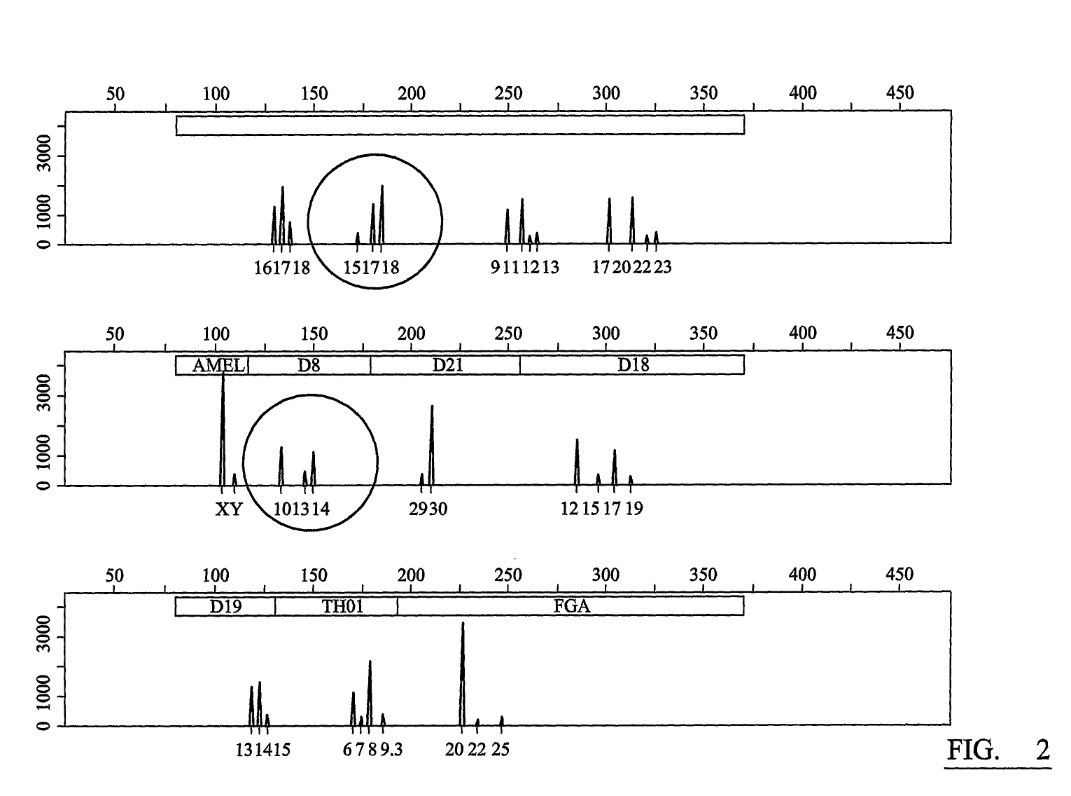

FIG. 2 is an example of a simulated profile;

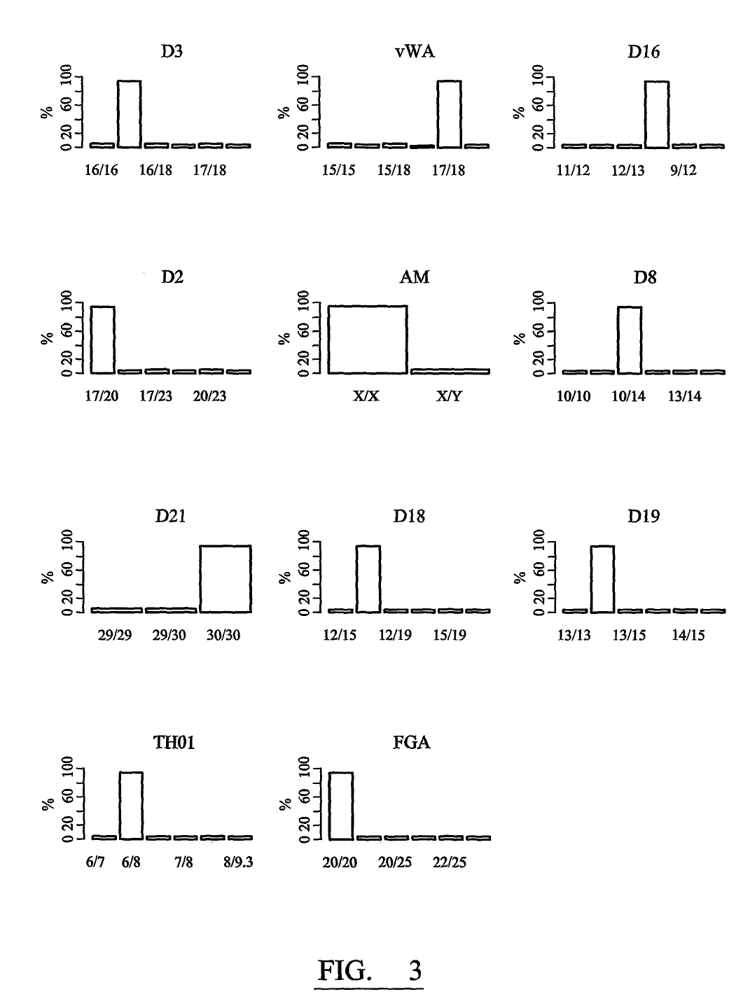

FIG. 3 illustrates the posterior probability of the major genotype by locus; and

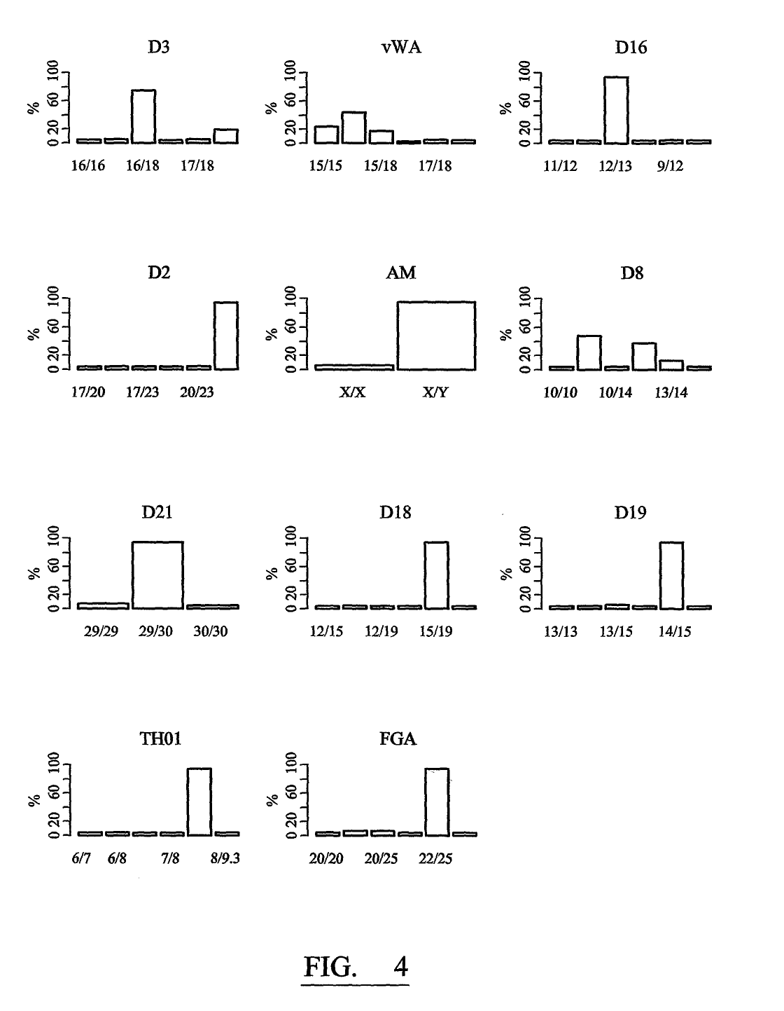

FIG. 4 illustrates the posterior probability of the minor genotype by locus.

When a DNA profile is obtained, that profile generally includes continuous quantitative information. Thus for a given size, the profile has a peak height or peak area, and for another given size, the profile has another peak area or peak height and so on.

Using the technique described in Gill et al. “Interpreting simple STR mixtures using allelic peak areas”, For. Sci. Int. 91 (1998) 41-53 it is possible to resolve a two person mixture. This technique has been more fully implemented in the Pendulum software product of The Forensic Science Service and is detailed in Bill et al. “Pendulum—A guideline based approach to the interpretation of STR mixtures”, For. Sci. Int. 148 (2005) 181-189. The technique is a rule based optimisation system and it works deterministically. The result of the full implementation to a profile is a list of 500 best results. However, in such an approach and in other prior art approaches, it is not possible to rank these results in a probabilistic manner. As such there is no assessment of the worth of the information returned.

### OVERVIEW

The present invention has a different approach. Firstly, a prior probability distribution, prior, is assigned to the genotypes. The likelihood function, likelihood, is then evaluated. From this prior and the likelihood a posterior probability distribution, posterior, can then be obtained. A variety of approaches can then be taken to sample the posterior. The distribution of the sample values can then be used to inform on the genotype.

The determining of the likelihood function is greatly assisted by the use of a graphical model as this provides useful structure to a complex consideration. By using a goodness of fit statistic, with the χ² distribution, it is possible to model the likelihood of the data given a mixing proportion and a genotype combination for the major and minor contributors. This likelihood along with some prior assumptions then allows a Monte Carlo Markov Chain method to be developed for sampling from the full posterior probability distribution of the genotypes, mixing proportions and associated hyper-parameters. A Metropolis-Hastings MCMC sampler in particular may be used. The sampling in turn allows probabilistic assessments on the genotype of the major or minor contributor to be made. As a result the approach provides posterior probability assessments of the most probable genotypes and a likely range for the mixing proportion.

### Graphic Model

An important part of the new approach is the use of a graphical model for the issues. In FIG. 1 a graphical model for a two person mixture is provided. Breaking down and presenting the position in this way allows a determination of the structure of the problem, before having to assess the quantitative issues in what is a complex stochastic system.

The graphical model has two main components. The first, nodes, represent variables. The second, directed edges, extend between nodes and represent the direct influence of one node (variable) on another. Direct edges extend from a parent node to a child node. No direct edges returning to the starting node are allowed. Nodes may be either constant nodes or be stochastic nodes. Constant nodes are fixed by the graphical model design and are always founder nodes; they may have child nodes, but do not have parent nodes. Stochastic nodes are variables and are given a distribution. They may have child and/or parent nodes. In FIG. 1, the constant nodes are shown as rectangles and the stochastic nodes are shown as circles.

The full description of the graphical model is provided below and includes details of the priors applied.

1. α, β hyper-parameters of the beta distribution placed on mₓ. each has a Gamma prior with shape parameter 1 and scale parameter 1,000, i.e. α, β˜Γ(1,1000).
2. mₓ the global mixing proportion. 100(1−mₓ)% of the mixture comes from the major contributor and 100mₓ% comes from the minor contributor. A a beta distribution is used to model mₓ with parameters α and β, i.e. mₓ˜βeta(α,β). However mₓ is scaled between 0.02 and 0.48.
3. σₘ_{ₓ} the standard deviation of the locus mixing proportion. The standard deviation on a given mixing proportion is about 3.5%, so σₘ_{ₓ} is fixed at 0.035.
4. γ, α the parameters of a beta distribution for the locus specific mixing proportion mₓₗ. These quantities are determined by the values of mₓ and σₘ_{ₓ} in the following way. For a given value of mₓ, qᵤₚₚₑᵣ=mₓ+2.32σₘ_{ₓ} is calculated, and then a golden section search is used to find γ and δ such that the mode of the beta distribution with these parameters is mₓ and the 0.99 quantile of the distribution is qᵤₚₚₑᵣ.
5. mₓₗ the locus specific mixing proportion, where l=l, . . . , nₗ and nₗ is the number of loci. The mixing proportion is allowed to vary from locus to locus according to a beta distribution with parameters γ, δ, i.e. mₓₗ˜βeta(γ,δ).
6. Gᵢ the genotype of the major and the minor contributor. Gᵢ is a 4×n_{L} array with each row consisting of four integers ranging from 1 to 4. The range of the integers will depend on the number of peaks observed at the locus, e.g. if there is only one peak then the entries will all have value 1, if there are two peaks then the entries can have value 1 or 2 etc. This means that the genotype of each contributor is specified as the peak that contributor contributed alleles to. The distributions for Gᵢ are locus specific and dependent on the number of peaks observed at that locus. Uniform probability is assigned to each combination of peaks. This means the prior distribution for Gᵢ is a discrete uniform prior over the space of allowable combinations.
7. {tilde under (φ)}ₗ the observed peak area(s) (or height(s)) at a locus. {tilde under (φ)}ₗ is a vector of length. It is assumed that {tilde under (φ)}ₗ it has a multivariate Normal distribution (MVN) with mean vector

$${{μ}_{l}}_{\sim} = \frac{{φ}_{l □}}{2} {( {m}_{xl} , {m}_{xl} , 1 - {m}_{xl} , 1 - {m}_{xl} )}^{′ }$$

and diagonal covariance matrix Σₗ=σₗ²I₄ where

$${φ}_{l □} = {\sum }_{i} {φ}_{li} .$$

The value of σₗ² is not important as this because it is not used direction. This assumption allows us to assume a χ² distribution for X²
8. X² the chi-squared distance of the observed data from expectation under the assumption that the mixing proportion for each locus is known.

$${X}^{2} = {\sum }_{l = 1}^{{n}_{l}} {\sum }_{i = 1}^{4} \frac{{( {φ}_{{lG}_{li}} - {E}_{li} )}^{2}}{{E}_{li}}$$

φ_{lG}_{ₗ} is the peak at the lth locus to which the major contributor is assumed to have contributed to for i=1, 2 and to which the major contributor is assumed to have contributed to for i=3, 4. Eₗᵢ is the expected peak area which is calculated as

$${E}_{li} = E [ {φ}_{li} \vert {m}_{xl} , {G}_{li ; minor} ] = \frac{{m}_{xl} {φ}_{l □}}{2}$$

for the each of the minor peaks and

$${E}_{li} = E [ {φ}_{li} \vert {m}_{xl} , {G}_{li ; major} ] = \frac{( 1 - {m}_{xl} ) {φ}_{l □}}{2}$$

for the major peaks

Some details of note within the graphical model are that:—

a) the true genotype is independent of any of the factors under consideration in the model;
b) the mixing proportion for all the loci, m, depends only on two hyper-parameters α and β;
c) the mixing proportion at each locus is conditionally dependent on two parameters γ and δ which are dependent on the mixing proportion for all loci, mₓ, the standard deviation of a mixing proportion at a locus;
d) the observed peak areas are dependent on the genotype and the mixing proportions at each locus;
e) the κ² statistic X² is dependent on the peak areas and the mixing proportions at each locus.

It should also be noted that it is assumed that there is an overall mixing proportion, with conditionally independent mixing proportions at each locus; the priors placed on each genotype are assumed to be uniform; the peak area is evaluated via a chi-squared distribution.

### Posterior Probability Distribution

Pendulum attempts to find the mixing proportion (or weight) associated with the minor contributor and the genotype combination that minimizes the squared distance between the observed areas and the expected areas. By letting mₓ be the mixing proportion, then for a given combination Gᵢ, and mₓ, the expected values are defined as:

$$E [ {φ}_{i} \vert {m}_{x} , {G}_{i ; minor} ] = \frac{{m}_{x} {φ}_{□}}{2}$$

for the each of the minor peaks and

$$E [ {φ}_{i} \vert {m}_{x} , {G}_{i ; major} ] = \frac{( 1 - {m}_{x} ) {φ}_{□}}{2}$$

for the major peaks
If Eᵢ is the expected area of peak i, then Pendulum attempts to find an mₓ such that

$${\sum }_{i} {( {φ}_{i} - {E}_{i} )}^{2}$$

is minimized.

In order to make a probabilistic interpretation of a particular combination, the ability to model the distribution of this distance measure is needed. There are several difficulties associated with this task. Firstly the underlying distribution of the area data is unknown, which in turn makes it difficult to model the distribution of the distance measure. Secondly, this distance measure gives more weight to loci with more peak area. Whilst this second problem may be remedied by scaling the peak areas so that they sum to one at each locus, such scaling can make modelling even more difficult.

In the present invention, the peak areas at a locus are modelled by a multivariate Normal (MVN) distribution, with mean vector

$${μ}_{\sim} = \frac{{φ}_{□}}{2} {( {m}_{x} , {m}_{x} , 1 - {m}_{x} , 1 - {m}_{x} )}^{′}$$

and diagonal covariance matrix

Σ=σ²I₄.

The result of making this assumption is that the following statistic

$${X}^{2} = {\sum }_{i = 1}^{4} \frac{{( {φ}_{i} - {E}_{i} )}^{2}}{{E}_{i}}$$

will have a χ² distribution with 3 degrees of freedom. This result can be extended over multiple loci, and the resulting statistic will have 4n₁−l degrees of freedom where l is the number of loci. This helps with the probabilistic assessment, because a likelihood function for the genotype Gᵢ and the mixing proportion mₓ, given the peak area information {tilde under (φ)} may be formulated which is a precursor to a tractable Bayesian method.

The true usefulness of a graphical model becomes apparent when it comes to writing the joint density function. For a given graph with node set V, can be expressed as

$$p ( {x}_{\sim} ) = {\prod }_{v ∈ V} p ( {x}_{v} \vert {x}_{pa ( v )} )$$

This expression in effect says mathematically that the joint density probability may be expressed as a product of the conditional densities of each variable given their parents in the graph. For the graph in FIG. 1, the joint density is:

$$f ( {X}^{2} , {φ}_{\sim} , {{m}_{\sim}}_{xl} , γ , δ , {m}_{x} , α , β , {G}_{i} ) = f ( {X}^{2} \vert {φ}_{\sim} , {{m}_{\sim}}_{xl} , {G}_{i} ) {\prod }_{l = 1}^{{n}_{l}} f ( {m}_{xl} \vert γ , δ ) f ( {m}_{x} \vert α , β ) f ( {G}_{i} ) f ( α ) f ($$

Note that there is no explicit term for f({tilde under (φ)}|Gᵢ, {tilde under (m)}ₓₗ) because this is evaluated “by proxy” by f(X²|{tilde under (φ)},{tilde under (m)}ₓₗ,Gᵢ).

Whilst the full density could be considered, it is of far less interest than the conditional density of the genotypes (and perhaps some of the other parameters given the data).

Bayes Theorem allows this to be to calculated as

$$f ( {G}_{i} , {m}_{x} , {θ}_{\sim} \vert {φ}_{\sim} ) = \frac{f ( {G}_{i} , {m}_{x} , {θ}_{\sim} \vert {φ}_{\sim} )}{f ( {φ}_{\sim} )}$$

where {tilde under (θ)}=({tilde under (m)}ₓₗ, γ, δ, α, β). The density of the data, f({tilde under (φ)}), is never known and so this equation can be rewritten as

$$f ( {G}_{i} , {m}_{x} , {θ}_{\sim} \vert {φ}_{\sim} ) = \frac{f ( {φ}_{\sim} \vert {G}_{i} , {m}_{x} , {θ}_{\sim} ) f ( {G}_{i} , {m}_{x} , {θ}_{\sim} )}{\int f ( {φ}_{\sim} \vert {G}_{i} , {m}_{x} , {θ}_{\sim} ) f ( {G}_{i} , {m}_{x} , {θ}_{\sim} ) \cdot d {φ}_{\sim}}$$

The integral on the denominator of this equation is very difficult to calculate exactly. However, it can by estimated with one or more Markov-Chain Monte Carlo (MCMC) methods.

A Metropolis-Hastings sampler, Metropolis et al., “Equations of state calculations by fast computing machines”, J. Chem. Phys. 21:1087-1092 (1953) and Hastings, “Monte Carlo sampling methods using Markov chains and their application”, Biometrika 57:97-109 (2004), provides a method for sampling from the posterior distribution of Gᵢ, mₓ, {tilde under (θ)}, given the data, {tilde under (φ)}. A simplified sampler for one locus can be defined as follows:

### Metropolis-Hastings Sampler

1. Randomly select an initial genotype G, and an initial mixing proportion mₓ

2. Calculate the log likelihood, l(G,mₓ|{tilde under (φ)}) using G and mₓ

3. Repeat the following as many times as desired
a. Select a new genotype G′, and a new mixing proportion mₓ′
b. Calculate the log likelihood, l′(G′,mₓ′|{tilde under (φ)}) using G′ and mₓ′
c. Generate u˜U[0,1]
d. If log (u)≦min(0,l′(G′,mₓ′|{tilde under (φ)})−l(G,mₓ|{tilde under (φ)})) then
   i. store G′ and mₓ′
   ii. let G=G′, mₓ=mₓ′,and l(G,mₓ|{tilde under (φ)})=l(G′,mₓ′|{tilde under (φ)})

The resulting stored sample, after a sufficient period, will be a sample from the full posterior distribution of G and mₓ given {tilde under (φ)}. This means the posterior probability density of the mixing proportion can be estimated by getting a density estimate of the stored values of mₓ and the posterior probability of the genotypes can estimated by counting how many times each one occurred.

This idea has been extended for multiple loci and incorporates extra terms in the graphical model. In this case the Metropolis-Hastings sampler provides a method for sampling from the posterior distribution of Gᵢ, mₓ, {tilde under (m)}ₓₗ, γ, δ, α, β, given the data, {tilde under (φ)}. A generalized sampler for multiple loci locus can be defined as follows:

### Metropolis-Hastings Sampler

1. Randomly select an initial genotype G, and initial values for mₓ,{tilde under (m)}ₓₗ, γ, δ, α and β
2. Calculate the log likelihood, l(G,{tilde under (θ)}|{tilde under (φ)}) where {tilde under (θ)}=(mₓ,{tilde under (m)}ₓₗ, γ, δ, α, β)
3. Repeat the following as many times as desired
   a. Select a random locus l,l˜U[1, . . . , n_{L}]
   b. Select a new genotype Gₗ′ at locus l, to give a new genotype G′
   c. Select new values for {tilde under (θ)}′=(mₓ′, {tilde under (m)}ₓₗ′, γ′, δ′, α′, β′)
   d. Calculate the log likelihood, l′(G′,{tilde under (θ)}′|{tilde under (φ)})
   e. Generate u˜U[0,1]
   f. If log (u)≦min(0,l′(G′,{tilde under (θ)}′|{tilde under (φ)})−l(G,{tilde under (θ)}|{tilde under (φ)})) then
      i. store G′ and {tilde under (θ)}ₓ′
      ii. let G=G′, {tilde under (θ)}={tilde under (θ)}′, and l(G,{tilde under (θ)}|{tilde under (φ)})=l′(G′,{tilde under (θ)}′|{tilde under (φ)})

The resulting stored sample, after a sufficient period, will be a sample from the fall posterior distribution of G and {tilde under (θ)} given {tilde under (φ)}. This means the posterior density of the model parameters can be estimated by getting a density estimate of the stored values of {tilde under (θ)} and the posterior probability of the genotypes can be estimated by counting how many times each one occurred.

### Experimental

To investigate the approach, experimental data was considered. Whilst this could be achieved through actual sample analysis, the applicant used the approach detailed in UK Patent Application No's 0426579.9 filed 3 Dec. 2004 and/or 0506673.3 filed 1 Apr. 2005 and/or PCT/GB2005/004641 filed 5 Dec. 2005, and/or Gill et al. “A graphical simulation model of the entire DNA process associated with the analysis of short tandem repeat loci.”, Nucleic Acids Research 33(2) (2005) 632-643 to generate a 5:1 proportioned mixture with known genotypes.

In one case, the simulation approach was applied with settings: loci 11, 54 diploid input cells (N=54), standard 28 cycle PCR (n_{cycles}=28), extraction efficiency of 60% (π_{extraction}=0.6), an aliquot of 20 μL per 66 μL was pipetted from the solution (π_{aliquot}=20/66) and the PCR efficiency was 80% (π_{PCReff}=0.8). In the initial consideration, the effects of stutter and dropout were discounted, but the approach could take these into account too and other effects such as preferential amplification. In a second case, the number of diploid input cells was changed to 270. Combined this gives 270:54 or 5:1.

Using a set of published Caucasian allele frequencies two 10 locus profiles were generated. One was designated male, Amelogenin to XY, and the other was designated female, Amelogenin to XX. An example of the simulated profile is shown in FIG. 2.

Combined, and together with the simulated peak area/height information, this produced a Pendulum input file. Reading this to a C++ programme (MCMC-Pendulum), the programme was allowed to run until 97,500 had been accepted. The acceptance rate was between 5 and 6 per 10,000 proposals. The first 7,500 iterations were discarded and every 9^{th} observation was sampled to give a final sample size of 10,000. Such a sampling rate was seen as negating any correlation present between successive proposals.

FIG. 3 illustrates the posterior probability of the major genotype by locus. The true genotype for the major occurs 5356 times in the sample of 10,000. The posterior probability of the major genotype is 0.2135 and it has the highest posterior probability. Indeed the probability is 17 times higher than the next possibility (probability 0.0315).

FIG. 4 illustrates the posterior probability of the minor genotype by locus. In the case of two loci, vWA and D8, the dominant posterior probability was not the true genotype. Each of these loci has three peaks, with a major who is heterozygous and with heterozygous imbalance between the two major peaks. At each locus, the remaining small peak is correctly selected as belonging to the minor, but the programme will score a heterozygous genotype with the second allele taken from the largest peak more highly than homozygous from the same small peak or heterozygous with the second largest peak. The posterior probability of the true minor genotype is 0.0234 and it has the 6^{th} highest posterior probability. This compares with the most likely having a posterior probability of 0.0835. Thus the top combination is roughly 2.8 times more likely than the true combination. In practice, a threshold can be used to define a cut off where faith in the genotypes no longer applies. In this example, if combinations that were no more than 10 times less likely than the top were taken, then that would give 16 possibilities.

The above experimentation demonstrates that a method for probabilistically resolving mixed DNA profiles into a major and minor component has been achieved. Because the method is set up in a Bayesian framework it also allows inferences about the parameters which are believed to drive the mixing process to be made and a probabilistic assessment of the genotypes to be produced. This is an advance over the previous methodology.

To address the problem of heterozygous balance—heterozygous balance (Hb) is the phenomenon whereby there is a difference in the peak heights (and areas) of a heterozygous genotype even though the genetic material comes from one person—an expansion of the graphical model and hence of the likelihood consideration can be made. The technique of UK Patent Application No 0426579.9 filed 3 Dec. 2004 and/or 0506673.3 filed 1 Apr. 2005 and/or PCT/GB2005/004641 filed 5 Dec. 2005 and/or Gill et al “A graphical simulation model of the entire DNA process associated with the analysis of short tandem repeat loci.”, Nucleic Acids Research 33(2) (2005) 632-643 can be used to simulate a probability density function for Hb and this could be incorporated in the graphical model. The net result of this is that it would add some flexibility to the method in situations like those at locus vWA where the preferred genotype for the minor contributor is 15/17 as opposed to the true genotype of 15/18. 15/17 is favoured because there is more peak area in the peak 17 than 18. Inclusion of an Hb term would allow the possibility that 18 may have had more area if it wasn't for Hb.

In a similar way the method could also be extended to include stutter, preferential amplification, artefacts and more than two contributors to the mixture.

1. A method of investigating a sample, the sample being a mixture of DNA arising from more than one source, the method including:
analysing the sample to obtain indications of the DNA present in the sample;
assigning a prior probability distribution to the indications;
considering the likelihood function;
establishing a posterior probability distribution for the indications.

2. A method according to claim 1 wherein the method includes analysing the sample to obtain a genotype for the DNA present in the sample, assigning a prior probability distribution to the genotype, considering the likelihood function and establishing a posterior probability distribution for the genotype.

3. A method according to claim 1 wherein the method includes assigning a prior probability distribution to the genotype obtained from analysis, considering the likelihood function and establishing a posterior probability distribution for the indications.

4. A method according to claim 1, wherein the prior probability distribution and the likelihood function is used to establish the posterior probability distribution.

5. A method according to claim 1, wherein provides a probabilistic assessment on the genotype of the major or minor contributor.

6. A method according to claim 1, wherein the method provides posterior probability assessments of the most probable genotypes and/or a likely range for the mixing proportion.

7. A method according to claim 1, wherein the posterior probability distribution informs on the probability of one or more indications or genotypes.

8. A method according to claim 7, wherein the posterior probability distribution informs by sampling the posterior probability distribution.

9. A method according to claim 8, wherein the distribution of the sample values obtained by sampling is used to inform on the indication and/or genotype.

10. A method according to claim 8, wherein the sampling is provided by a Monte Carlo Markov Chain method.

11. A method according to claim 1, wherein the considering of the likelihood function and/or the establishment of the factors involved in the likelihood function uses a graphical model.

12. A method according to claim 11, wherein the likelihood function and/or graphical model, includes a model of the distribution of the distance measure between the expected and observed information.

13. A method of investigating a sample, according to claim 1, wherein the sample being a mixture of DNA arising from more than one source, the method including:
analysing the sample to obtain indications of the DNA present in the sample;
establishing one or more possible genotypes for the DNA sample;
establishing a probabilistic measure of the possible genotype being the genotype of the sample; and
considering only those of the one or more possible genotypes for the DNA sample which have a probabilistic measure beyond a threshold against one or more records of genotypes, such as a database.
